(19) United States
(12) Patent Application Publication
(10) Pub. No.: US 2022/0301713 A1
(43) Pub. Date: Sep. 22, 2022

(54) SYSTEMS AND METHODS FOR DISEASE AND TRAIT PREDICTION THROUGH GENOMIC ANALYSIS

(71) Applicant: SALK INSTITUTE FOR BIOLOGICAL STUDIES, La Jolla, CA (US)

(72) Inventors: Debha AMATYA, La Jolla, CA (US); Fred H. GAGE, La Jolla, CA (US)

(21) Appl. No.: 17/632,658

(22) PCT Filed: Jul. 10, 2020
(86) PCT No.: PCT/US2020/041725

§ 371 Date: Feb. 3, 2022

Related U.S. Application Data
(60) Provisional application No. 62/873,802, filed on Jul. 12, 2019.

(51) Int. Cl. G16H 50/20; G16H 50/30

(52) U.S. Cl. CPC G16H 50/20; G16H 50/30

(57) ABSTRACT

A method to diagnose hereditary diseases or traits, is provided. The method includes receiving a genomic characterization for a patient, applying a variant filter against the genomic characterization to reduce a pool of relevant variants for the patient to form a filtered genomic characterization of the patient, and forming a vector in a multidimensional space, the vector including a score associated with each variant for each gene in the filtered genomic characterization of the patient. The method also includes transforming the vector to a reduced vector, and inputting the reduced vector in an analytical model to diagnose a presence of the hereditary diseases or traits, including genomic characterizations of each individual in a population of individuals, each genomic characterization indicative of a relative presence of the hereditary diseases or traits in a specific individual in the population of individuals. A system to perform the above method is also provided.

### FIELD

The embodiments provided herein are generally related to systems and methods for analysis of genomic nucleic acids and classification of genomic features.

### BACKGROUND

A central goal of biomedical genomic analysis is to elucidate the relationship between disease phenotypes and their genetic underpinnings. For certain conditions, such as rare monogenic disorders or cancer, genome sequencing has already proven to be clinically useful in risk assessment, diagnosis, and treatment selection. However, such success has remained more elusive in more common but complex conditions, which are often influenced by environmental factors and characterized by distributed genetic risk. Modeling how risk is integrated across the genome remains a critical challenge for complex heritable disorders.

Despite the existence of big genomic data and machine learning tools, automated genomic classification has not yet demonstrated robust and reproducible results for neuropsychiatric disease prediction. Genetic heterogeneity, low statistical power, and data dimensionality are common issues encountered in such studies. A vector cast in primary sequence or variant space may have a prohibitively large dimensionality, whereas smaller representations may not sufficiently encode the complexity of the disease signature.

As such, there remains a need for improved diagnosis based on computationally efficient and biologically relevant representations and classification of an individual genome that balance dimensionality with biological information content.

### SUMMARY

In accordance with various embodiments, a computer-implemented method is provided to diagnose hereditary diseases or traits. The computer-implemented method may comprise receiving a genomic characterization for a patient. The computer-implemented method may comprise receiving a risk feature that correlates with a presence of the hereditary diseases or traits, wherein an analytical model identified the risk feature when the analytical model was being trained using a cross-validation based on vectorized genomic characterizations. The training set and the validation set are portions of vectorized genomic characterizations of each individual in a population of individuals with a known presence or absence of the hereditary disease or traits. The computer-implemented method may comprise diagnosing the hereditary disease or traits of the patient based on the risk feature.

The computer-implemented method may comprise receiving a genomic characterization for a patient. The computer-implemented method may comprise applying a variant filter against the genomic characterization to reduce a pool of relevant variants for the patient to form a filtered genomic characterization of the patient. The computer-implemented method may comprise forming a vector in a multidimensional space, the vector including a score associated with each variant for each gene in the filtered genomic characterization of the patient. The computer-implemented method may comprise transforming the vector to a reduced vector using a dimensionality reduction technique. For example, the dimensionality reduction technique may comprise one of a visualization tool for differentiating a vector projection in a reduced dimensional space according to a pre-selected boundary, or a selection of a higher variance gene subset meeting a threshold, the threshold being indicative of variants having greater association to a disease or trait than variants not meeting the threshold. The computer-implemented method may comprise inputting the reduced vector in an analytical model to diagnose a presence of the hereditary diseases or traits, wherein the analytical model is trained using a cross-validation of a training set, the training set comprising genomic characterizations of each individual in a population of individuals, each genomic characterization indicative of a relative presence of the hereditary diseases or traits in a specific individual in the population of individuals.

In accordance with various embodiments, a non-transitory computer-readable medium is provided to diagnose hereditary diseases or traits. The non-transitory computer-readable medium store compute instructions that, when executed by a processor, cause the processor to receive a genomic characterization for a patient. The non-transitory computer-readable medium store compute instructions that, when executed by a processor, cause the processor to receive or obtain a risk feature that correlates with a presence of the hereditary diseases or traits, wherein an analytical model identified the risk feature when the analytical model was being trained using a cross-validation based on vectorized genomic characterizations. The training set and the validation set are portions of vectorized genomic characterizations of each individual in a population of individuals with a known presence or absence of the hereditary disease or traits. The non-transitory computer-readable medium store compute instructions that, when executed by a processor, cause the processor to diagnose the hereditary disease or traits of the patient based on the risk feature.

In accordance with various embodiments, a non-transitory computer-readable medium is provided to diagnose hereditary diseases or traits. The non-transitory computer-readable medium store compute instructions that, when executed by a processor, cause the processor to receive a genomic characterization for a patient. The non-transitory computer-readable medium store compute instructions that, when executed by a processor, cause the processor to apply a variant filter against the genomic characterization to reduce a pool of relevant variants for the patient to form a filtered genomic characterization of the patient. The non-transitory computer-readable medium store compute instructions that, when executed by a processor, cause the processor to form a vector in a multidimensional space, the vector including a score associated with each variant for each gene in the filtered genomic characterization of the patient. The non-transitory computer-readable medium store compute instructions that, when executed by a processor, cause the processor to transform the vector to a reduced vector using a dimensionality reduction technique. For example, the dimensionality reduction technique may comprise one of a visualization tool for differentiating a vector projection in a reduced dimensional space according to a pre-selected boundary, or a selection of a higher variance gene subset meeting a threshold, the threshold being indicative of variants having greater association to a disease or trait than variants not meeting the threshold. The non-transitory computer-readable medium store compute instructions that, when executed by a processor, cause the processor to input the reduced vector in an analytical model to diagnose a presence of the hereditary diseases or traits, wherein the analytical model is trained using a cross-validation of a training set, the training set comprising genomic characterizations of each individual in a population of individuals, each genomic characterization indicative of a relative presence of the hereditary diseases or traits in a specific individual in the population of individuals.

In accordance with various embodiments, a system is provided to diagnose hereditary diseases or traits. The system may comprise a memory storing instructions; and one or more processors configured to execute the instructions to cause the system to receive a genomic characterization for a patient. The instructions may be caused to receive or obtain a risk feature that correlates with a presence of the hereditary diseases or traits, wherein an analytical model identified the risk feature when the analytical model was being trained using a cross-validation based on vectorized genomic characterizations. The training set and the validation set are portions of vectorized genomic characterizations of each individual in a population of individuals with a known presence or absence of the hereditary disease or traits. The instructions may be caused to diagnose the hereditary disease or traits of the patient based on the risk feature.

In accordance with various embodiments, a system is provided to diagnose hereditary diseases or traits. The system may comprise receiving a genomic characterization for a patient. The system may comprise applying a variant filter against the genomic characterization to reduce a pool of relevant variants for the patient to form a filtered genomic characterization of the patient. The system may comprise forming a vector in a multidimensional space, the vector including a score associated with each variant for each gene in the filtered genomic characterization of the patient. The system may comprise transforming the vector to a reduced vector using a dimensionality reduction technique. For example, the dimensionality reduction technique may comprise one of a visualization tool for differentiating a vector projection in a reduced dimensional space according to a pre-selected boundary, or a selection of a higher variance gene subset meeting a threshold, the threshold being indicative of variants having greater association to a disease or trait than variants not meeting the threshold. The system may comprise inputting the reduced vector in an analytical model to diagnose a presence of the hereditary diseases or traits, wherein the analytical model is trained using a cross-validation of a training set, the training set comprising genomic characterizations of each individual in a population of individuals, each genomic characterization indicative of a relative presence of the hereditary diseases or traits in a specific individual in the population of individuals.

In accordance with various embodiments, a computer-implemented method is provided to train an analytical model for diagnosis of hereditary diseases or traits. The computer-implemented method may comprise receiving a genomic characterization of each individual in a population of individuals, the population of individuals selected to form a sampling set of a relative manifestation of a disease or trait. The computer-implemented method may comprise forming a variant filter against the genomic characterization of each individual to obtain a reduced pool of variants, the reduced pool of variants meeting a threshold associated with the variant filter, indicative of variants having a greater association to a disease or trait than variants not meeting the threshold. The computer-implemented method may comprise forming a vector in a multidimensional space using the reduced pool of variants, the vector having scores associated with each variant for each gene in the genome characterization of each individual. The computer-implemented method may comprise transforming a vector to a reduced vector through a dimensionality reduction technique to reduce dimensionality of the vector. The computer-implemented method may comprise selecting a first portion of the reduced vectors, to form a training set and a second portion of the reduced vectors, to form a validation set. The computer-implemented method may comprise finding multiple coefficients in an analytical model by applying the analytical model to the first portion of the reduced vectors to match a known condition of the disease or trait for each individual in the training set. The computer-implemented method may comprise evaluating a performance of the analytical model by applying the analytical model to the second portion of the reduced vectors for each individual in the validation set.

In accordance with various embodiments, a non-transitory computer-readable medium is provided for storing instructions that, when executed by a processor, cause the processor to receive a genomic characterization of each individual in a population of individuals, the population of individuals selected to form a sampling set of a relative manifestation of a disease or trait. The non-transitory computer-readable medium store compute instructions that, when executed by a processor, cause the processor to form a variant filter against the genomic characterization of each individual to obtain a reduced pool of variants, the reduced pool of variants meeting a threshold associated with the variant filter, indicative of variants having a greater association to a disease or trait than variants not meeting the threshold. The non-transitory computer-readable medium store compute instructions that, when executed by a processor, cause the processor to form a vector in a multidimensional space, the vector having scores associated with each variant for each gene in the genome characterization of each individual. The non-transitory computer-readable medium store compute instructions that, when executed by a processor, cause the processor to transform a vector to a reduced vector through gene variance filtering to meet a threshold or dimensionality reduction. The non-transitory computer-readable medium store compute instructions that, when executed by a processor, cause the processor to select a first portion of the reduced vectors, to form a training set and a second portion of the reduced vectors, to form a validation set. The non-transitory computer-readable medium store compute instructions that, when executed by a processor, cause the processor to find multiple coefficients in an analytical model by applying the analytical model to the first portion of the reduced vectors to match a known condition of the disease or trait for each individual in the training set. The non-transitory computer-readable medium store compute instructions, when executed by a processor, cause the processor to evaluate a performance of the analytical model by applying the analytical model to the second portion of the reduced vectors for each individual in the validation set.

In accordance with various embodiments, a system is provided to train an analytical model for diagnosis of hereditary diseases or traits. The system may comprise receiving a genomic characterization of each individual in a population of individuals, the population of individuals selected to form a sampling set of a relative manifestation of a disease or trait. The system may comprise forming a variant filter against the genomic characterization of each individual to obtain a reduced pool of variants, the reduced pool of variants meeting a threshold associated with the variant filter, indicative of variants having a greater association to a disease or trait than variants not meeting the threshold. The system may comprise forming a vector in a multidimensional space, the vector having scores associated with each variant for each gene in the genome characterization of each individual. The system may comprise transforming a vector to a reduced vector through gene variance filtering to meet a threshold or dimensionality reduction. The system may comprise selecting a first portion of the reduced vectors, to form a training set and a second portion of the reduced vectors, to form a validation set. The system may comprise finding multiple coefficients in an analytical model by applying the analytical model to the first portion of the reduced vectors to match a known condition of the disease or trait for each individual in the training set. The system may comprise evaluating a performance of the analytical model by applying the analytical model to the second portion of the reduced vectors for each individual in the validation set.

### BRIEF DESCRIPTION OF FIGURES

FIG. 1 illustrates a sequence of steps in a method for quantifying risk for hereditary diseases or traits, according to various embodiments.

FIG. 2 illustrates a dimensionality curve and a classification error curve in a feature space for modeling hereditary disease risk and trait prediction from a genome characterization, according to various embodiments.

FIG. 3 illustrates a sequence of steps in a method for processing variants in a genome characterization, according to various embodiments.

FIG. 4 illustrates a variant burden matrix for hereditary disease risk and trait prediction from a genome characterization, according to various embodiments.

FIG. 5 illustrates a principal component's plot of variant burden vectors, according to various embodiments.

FIG. 6A illustrates a vector pre-processing and training scheme, according to various embodiments.

FIG. 6B illustrates average model accuracy across cross-validation folds in a set of genomics data from the MSSNG database, according to various embodiments.

FIG. 6C illustrates classifier sensitivity and specificity of five different classification models through a representative receiver operating curve, according to various embodiments.

FIG. 6D illustrates classification accuracy performance of five classification models in another whole genome sequence dataset, according to various embodiments.

FIG. 6E illustrates classification specificity performance of five classification models in another set of independent ASD genomics data, according to various embodiments.

FIG. 6F illustrates another vector pre-processing and training scheme using both MSSNG vectors and SFARI vector, according to various embodiments.

FIG. 6G illustrates average model accuracy of five classification models using both MSSNG vectors and SFARI vectors, according to various embodiments.

FIG. 6H illustrates the receiver operating characteristic curves of the Naive Bayes model using both MSSNG vectors and SFARI vectors, according to various embodiments.

FIGS. 7A-E illustrate the extraction of salient genes for an exemplary hereditary disease, and the biological relevance of the extracted salient genes, according to various embodiments.

FIGS. 8A-8B are exemplary flow charts illustrating steps in methods for hereditary disease risk or trait assessment from a genetic characterization of an individual, according to various embodiments.

FIG. 9 is a flow chart illustrating steps in a method for training an analytical model for risk assessment of hereditary diseases or traits, according to various embodiments.

FIG. 10 is a block diagram that illustrates a computer system used to perform at least some of the steps and methods in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification and Appendix (provided below) describes various exemplary embodiments of systems, methods, and software for enhanced novelty detection. The disclosure, however, is not limited to these exemplary embodiments and applications or to the manner in which the exemplary embodiments and applications operate or are described herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular.

All publications mentioned herein are incorporated herein by reference for the purpose of describing and disclosing devices, compositions, formulations, and methodologies which are described in the publication and which might be used in connection with the present disclosure.

As used herein, the terms “comprise,” “comprises,” “comprising,” “contain,” “contains,” “containing,” “have,” “having,” “include,” “includes,” and “including” and their variants are not intended to be limiting, are inclusive or open-ended and do not exclude additional, unrecited additives, components, integers, elements, or method steps. For example, a process, method, system, composition, kit, or apparatus that comprises a list of features is not necessarily limited only to those features but may include other features not expressly listed or inherent to such process, method, system, composition, kit, or apparatus.

### Genome Characterization

In accordance with various embodiments herein, the systems, methods, and software are described for quantifying the risk of a hereditary disease or trait in a patient, including receiving a genomic characterization of each individual in a population of individuals selected to form a sampling set of a manifestation of disease or trait and using the genomic characterization to train analytical models for diagnosis of a presence of a hereditary disease or trait in a specific individual. In accordance with various embodiments herein, systems, methods, and software are described that obtain, provide, or receive genomic characterization such as genome sequence data, for example, whole genome sequencing data or partial genome sequence data. In accordance with various embodiments, genome characterization may also include vectorized genome data.

Non-limiting systems, methods, and software for genome sequencing include high throughput sequencing or next generation sequencing technologies (NGS) in which clonally amplified DNA templates and single DNA molecules are sequenced in a massively parallel fashion, such as pyrosequencing, DNA nanoball sequencing, sequencing-by-synthesis, sequencing by oligonucleotide probe ligation and real-time sequencing, or a combination thereof. In accordance with various embodiments, a combination of DNA nanoball sequencing and sequencing-by-synthesis can be used to obtain whole genome sequencing data as a genomic characterization for a patient.

### Analytical Models

In accordance with various embodiments herein, the systems, methods, and software are described for quantifying the risk of a hereditary disease or trait in a patient, including training an analytical model to classify or diagnose a presence or absence of a hereditary disease or traits. In accordance with various embodiments herein, systems, methods, and software are described that provide a technical solution to the technical problem of identifying a risk that patient may have a disease, and selecting a treatment for a condition based on a genomic characterization of the patient. In various embodiments, analytical models used herein may be a classification model or a machine learning model, such as a supervised learning model or an unsupervised learning model. Non-limiting examples of analytical models may include logistic regression, support vector machine, multilayer perceptron or neural network, Naïve Bayes, random forest, decision trees, k-nearest-neighbor, linear regression, classification trees, or a combination thereof.

In accordance with various embodiments, logistic regression may be selected to be used as an analytical model for classifying or diagnosing a presence or absence of a hereditary disease or traits, such as ASD. Logistic regression is a statistical model that may use a logistic function to model a binary dependent variable (binary regression) or a range of finite options (multinomial regression).

In accordance with various embodiments, support vector machine may be selected to be used as an analytical model for classifying or diagnosing a presence or absence of a hereditary disease or traits, such as ASD. A support-vector machine may construct a hyperplane or a set of hyperplanes in a high- or infinite-dimensional space. The support vector machine algorithm may be to find a hyperplane in an N-dimensional space (N—the number of features) that distinctly classifies data points. To separate the two classes of data points, there are many possible hyperplanes that could be chosen. One way may be to find a plane that has the maximum margin, i.e., the maximum distance between data points of both classes. Maximizing the margin distance may provide some reinforcement so that future data points can be classified with more confidence.

In accordance with various embodiments, neural network such as multilayer perceptron may be selected to be used as an analytical model for classifying or diagnosing a presence or absence of a hereditary disease or traits, such as ASD. A multilayer perceptron (MLP) is a class of feedforward artificial neural network (ANN). MLP may comprise more than one perceptron. MLP may comprise an input layer to receive the signal, an output layer that makes a decision or prediction about the input, and in between those two, an arbitrary number of hidden layers that are the true computational engine of the MLP. MLPs with one or more hidden layers may be capable of approximating any continuous function.

In accordance with various embodiments, Naïve Bayes may be selected to be used as an analytical model for classifying or diagnosing a presence or absence of a hereditary disease or traits, such as ASD. Naive Bayes methods may include a set of supervised learning algorithms based on applying Bayes' theorem with a “naive” assumption of conditional independence between every pair of features given the value of the class variable.

In accordance with various embodiments, random forest may be selected to be used as an analytical model for classifying or diagnosing a presence or absence of a hereditary disease or traits, such as ASD. Random forest algorithm may create decision trees on data samples and then obtain the prediction from each of them and finally select the best solution by means of voting. It may be an ensemble method which is better than a single decision tree because it may reduce the over-fitting by averaging the result.

### Genome Vectorization

In accordance with various embodiments herein, the systems, methods, and software are described for quantifying the risk of a hereditary disease or trait in a patient, including using genome vectorization to train sensitive and specific machine learning models, such as classification models. In accordance with various embodiments herein, systems, methods, and software are described that provide a technical solution to the technical problem of identifying a risk that patient may have a disease, and selecting a treatment for a condition based on a genomic characterization of the patient.

Embodiments as disclosed herein include genome classification techniques for risk assessment of hereditary disorders and other traits. To achieve this, various embodiments combine whole genome sequencing technology and machine-learning based on large trained sample sets. Many disorders, such as cancer, metabolic disorders, and neuropsychiatric illnesses, are genetic in nature. However, efforts to utilize genetic sequencing technology for clinical diagnosis have been challenged by the genetic complexity of most diseases. The biomedical science community has begun to identify the mechanistic role of genes, but an integrated understanding of genetic disorders remains a distant goal.

An alternative approach to bridging the gap between DNA and diagnosis is to feed large amounts of gene sequencing data (which already exists for many disorders) into machine-learning models that are capable of learning complex, multivariate gene mutation patterns that can be readily leveraged for disease diagnosis. However, this approach precludes a detailed mechanistic understanding of a given disease. This limitation may be redeemed by the rapidity, accuracy, cost, and potential clinical impact of genome classification. These advantages help understand why machine-learning has been applied to numerous other fields, e.g., fraud detection, autonomous vehicles, image search, and natural language processing.

In various embodiments, genome classification includes deep neural networks on a large cohort of certain hereditary disease genomes (e.g., autism spectrum disorder—ASD, and the like). Various embodiments achieve clinical grade performance for ASD diagnostic accuracy. Moreover, various embodiments include training new models to predict specific cognitive deficits or disease severity based on the patient genome. In a clinical setting, embodiments of genome classification techniques as disclosed herein may enable disease management by early (pre-symptom manifestation) detection or even prenatal diagnosis and early initiation of therapy (e.g., in the case of ASD). Based on the technical advances provided by embodiments as disclosed herein, patients may have higher probability for a neurotypical development. With similar effect, genome classification techniques can be applied to other hereditary diseases or related clinical-genetic applications, such as prognosis and treatment selection.

In various embodiments, genome classification techniques may be configured to provide mechanistic interpretations that yield novel scientific insights. For example, the input layer of a neural network as disclosed herein may correspond to genes or gene derivative features via a dimensionality reduction technique, such as principal component analysis (PCA) or singular value decomposition (SVD). In various embodiments, examination of gene activation at the input layer can serve to identify important genes or features that differentiate control and case subjects. Further, in various embodiments, the first hidden layer in the neural network may indicate activation patterns of gene/feature layer inputs.

In various embodiments, a final layer in the neural network may include a class probability prior to classification of each subject. Accordingly, in various embodiments, a disease risk may be determined based on the entropy of this probability distribution, for each patient genome. Using the risk estimation, high-risk or low-risk patients may be examined to identify putative risk or protective genetic features in a disease or trait. Comparison with the existing genome knowledge may yield novel mechanistic pathways, risk loci, or therapeutic targets in a given disorder or trait.

The use of a simple density plot of annotated variants across disease samples and controls has proven inadequate to distinguish between the two sets. Such simplistic approaches lack the resolution to separate the groups in a meaningful manner. What is desired is a method and a system that can quickly determine, upon analysis of a relatively simple set of risk factors, a distinction between a control sample and a diseased sample. Machine-learning models as disclosed herein provide a risk assessment, and support a clinical correlation between identified genetic risk features.

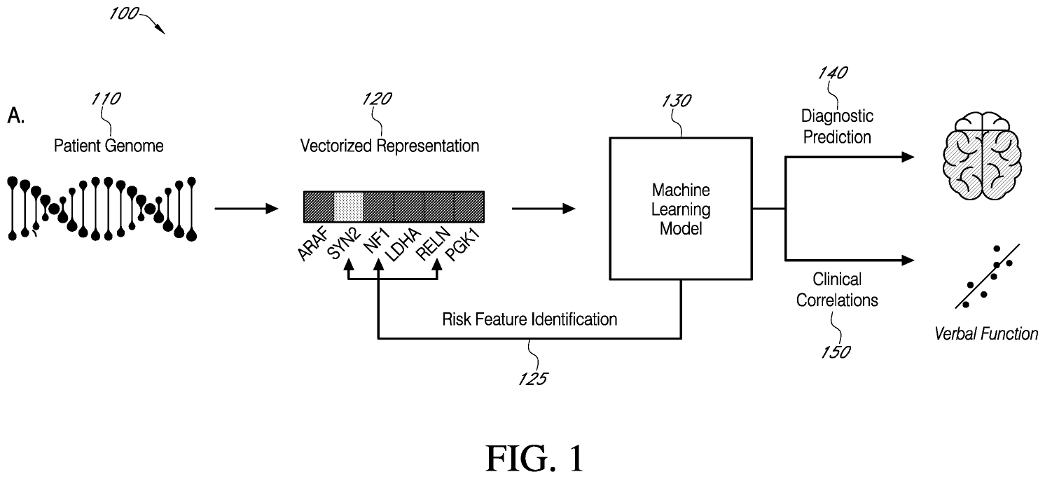

FIG. 1 illustrates a sequence of steps in a method 100 for quantifying risk for hereditary diseases or traits, according to various embodiments. The method includes an end-to-end solution for a machine learning analysis of the sequencing data of a hereditary disease. At the input, a dense, raw genome sequencing dataset 110 is transformed into a compact, vectorized representation 120 that is input to a machine learning model 130. The vectorized representation 120 may include specific gene variants (e.g., ARAF, SYN2, NF1, LDHA, RELN, and PGK1) that may be indicative of a high risk of developing the disease (e.g., ASD). The trained machine-learning model 130 may offer risk assessment or diagnostic prediction 140, and/or clinical correlation insight 150 (e.g., drugs and therapeutics) for consideration. The machine-learning model 130 may also be transparent in showing how genes are for prediction, and allow biological plausibility and mechanistic principles to be assessed. In various embodiments, a risk feature 125 may be provided that can include the set of variants SYN2, NF1, and RELN as illustrated for example, in the sequence (e.g., for ASD). The whole genome sequencing samples used to complete methods as disclosed herein may be stored in a database and remotely accessed online, e.g., the Autism Speaks database (MSSNG), and the like. The abundance of case and control samples allows for the full diversity of relevant genomic signatures to be learned by the machine-learning model 130. For example, in various embodiments (e.g., the MSSNG database), the total number of control and disease genomes may be in the thousands (e.g., 3,762 and 3,425 respectively, in the MSSNG, for a total of 7187 genomes from different individuals). In various embodiments, the inclusion of disease cases from both male and female genders is desirable, to allow for the generalization of results to both genders.

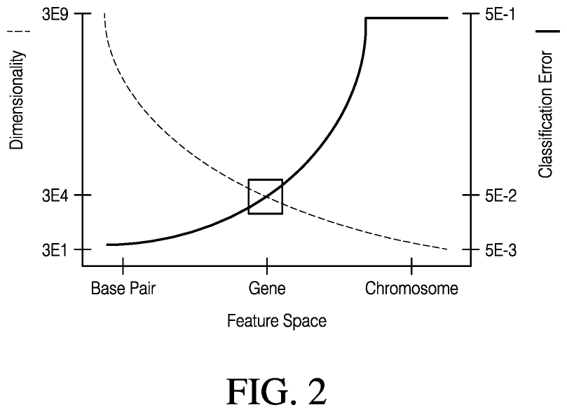

FIG. 2 illustrates a dimensionality curve and a classification error curve in a feature space for modeling hereditary disease risk and trait prediction from a genome characterization, according to various embodiments. The dimensionality and classification error curves illustrate that the choice of dimensional scale for vectorization impacts the predictive value of the data. A vectorization at the base pair level will likely encode all genetic risk but at a high computational cost. In contrast, a vector that summarizes the genome to chromosome scale features will be computationally efficient but too coarse to derive biological predictions from. Embodiments as disclosed herein use a gene-based scale classification that offers a good trade-off between computational efficiency and biological relevancy. A gene-based classification is desirable as the basis for genome vectorization because it is broad enough to reduce the dimensionality of the problem, yet specific enough to capture relevant disease-associated patterns.

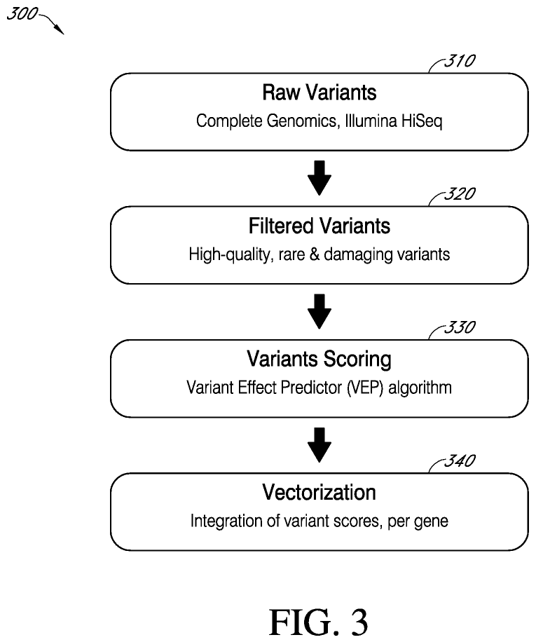

FIG. 3 illustrates a sequence of steps in a method 300 for processing variants in a genome characterization, according to various embodiments. In various embodiments, raw variants 310 (e.g., the whole genome) are filtered for quality criterion, as well as minor allele frequency and predicted or known deleteriousness to provide filtered variants 320. Various embodiments exclude variant calls present only in control samples (e.g., from healthy individuals), thereby focusing in on disease relevant variants only. Additionally, various embodiments only include variants when they had passed various filtering criteria for quality criterion, allele frequency (e.g., minor allele frequency ≤1%, 5%, 10%, 20%, 30%, 40%, 50%, or any range or value derivable therefrom) and effect (damage prediction, conservation, and known clinical). Accordingly, the filtered variants 320 may be high quality, rare, and damaging variants (e.g., likely associated with a disease such as ASD).

The filtered variants 320 may be scored to provide variants scoring 330 using, for example, a Variant Effect Predictor (VEP) tool. In various embodiments, the VEP tool categorizes the variants on a four-tier scale: “Modifier,” “Low,” “Moderate,” and “High” consequence. These labels may be converted to numeric values and averaged per gene for each individual, resulting in a gene-based variant burden vector. In various embodiments, a VEP tool includes a wide range of bioinformatics tools and databases to assess the impact of both coding and noncoding variation in sequencing data. VEP tools may be used to score the consequence of each annotated variant on a scale of 1 to 4, defined as follows: a score of 1 may be assigned to the “Modifier” variants, e.g., intergenic variants or minor regulatory region modifications. A score of 2 may be assigned to the “Low” variants, e.g., synonymous substitutions. A score of 3 may be assigned to the “Moderate” variants, e.g., missense mutations and in-frame insertions or deletions. Finally, a score of 4 may be assigned to the “High” variants, e.g., frameshift mutations or transcript ablations.

A vectorization step 340 for integrating variant scores, per gene, may include calculating an average VEP score for each gene for a given individual's annotated variants. Averaging may be performed to correct for differences in the number of variants across genes and subjects. For example, each subject variant burden vector may have a dimensionality of 30,676 genes for the MSSNG genomic data.

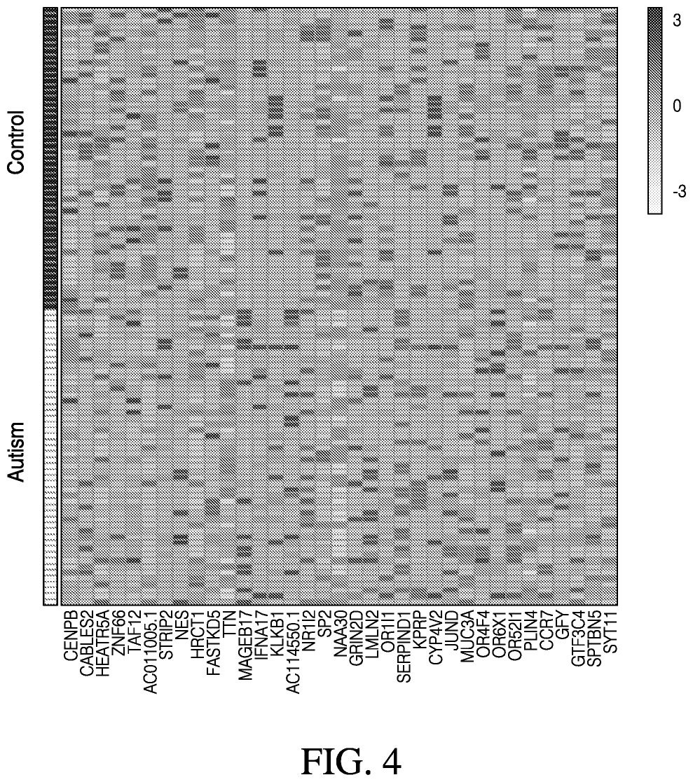

FIG. 4 illustrates a variant burden matrix for hereditary disease risk and trait prediction from a genome characterization, according to various embodiments. In the illustrated example, individual subject vectors were concatenated as rows to construct a 7,187×30,729 gene-based variant burden matrix. The partial display of the variant burden matrix offers a visualization of variant burden vectors as the rows of a 7,187×30,729 variant burden matrix. A small portion of this matrix is shown, with the i-th subject (row) and j-th gene burden (column) on a standardized scale. Rows corresponding to control subjects (e.g., healthy individuals) and disease-carrying subjects (e.g., individuals diagnosed with the ASD) are labeled. Embodiments as disclosed herein provide techniques to automatically distinguish group-based differences in the variant burden matrix.

To further reduce the dimensionality of the variant burden matrix, various embodiments may include an unbiased variance-filtering step or a dimensionality reduction step to select a pre-selected set of higher variance genes, such as higher variance genes in the top 10%, 20%, 30%, 40%, 50%, 60%, 70%, 80%, 90%, or any ranges or percentages derived therefrom. For example, a dimensionality reduction step may include selecting from the variant burden matrix those genes for which the variant score is higher than the median of the score distribution (for each gene, across all subjects), and therefore selecting a top half of higher variance genes. This would reduce the dimensionality of the variant burden matrix of FIG. 4, for example, to 7,187×15,338.

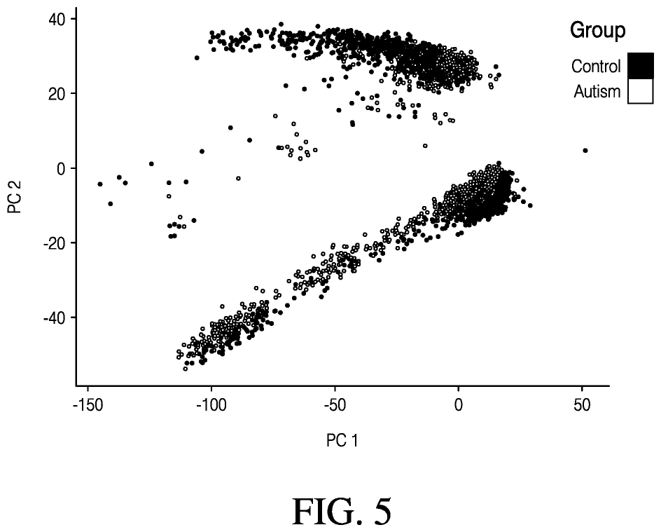

FIG. 5 illustrates a principal components plot of the variant burden vectors in the variant burden matrix, according to various embodiments. In order to more easily visualize group differences in the data, various embodiments may include a principal component analysis (PCA) step to reveal group separability in the first two principal components. The group of healthy individuals may be separated from the group of individuals diagnosed with the disease. Though the groups do not entirely form distinct clusters in this view, a classification boundary is apparent. The PCA plot may reveal other features, such as two large clusters in the plot, each including both control and disease samples, corresponding to differences in the genomic sequencing platform used.

### Training of Analytical Models (e.g., Machine Learning Models) Using Vectorized Genome

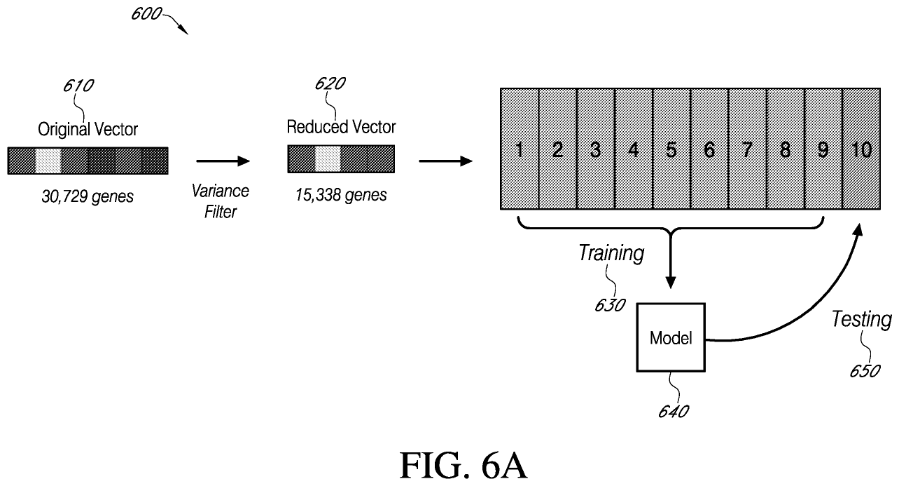

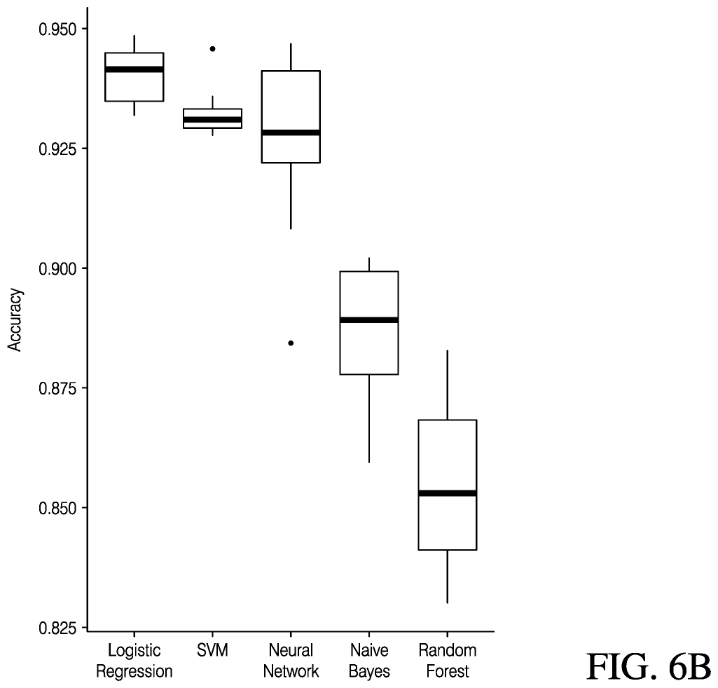

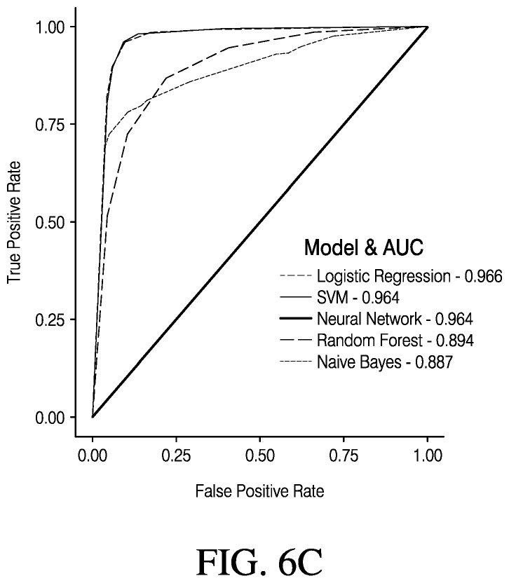

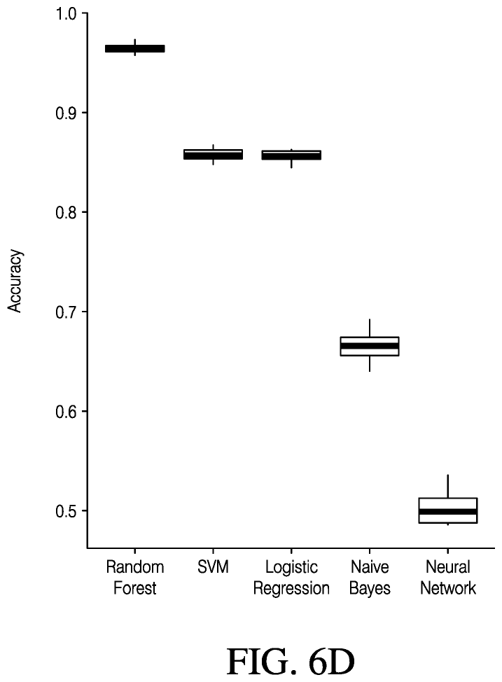

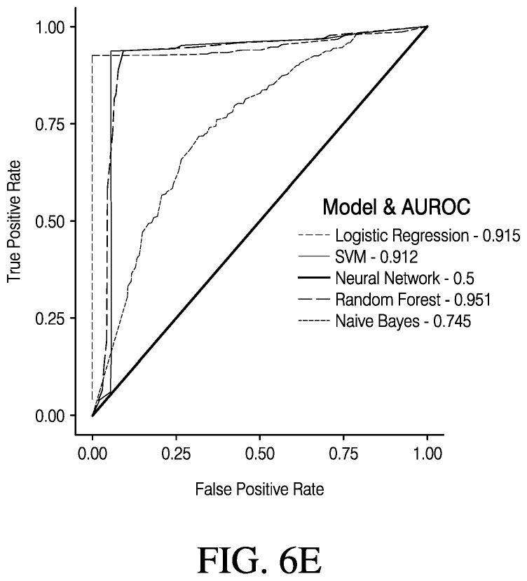

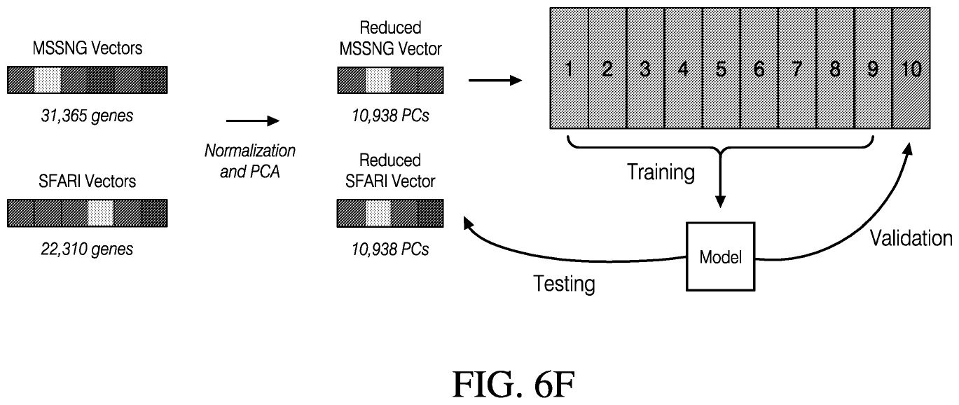

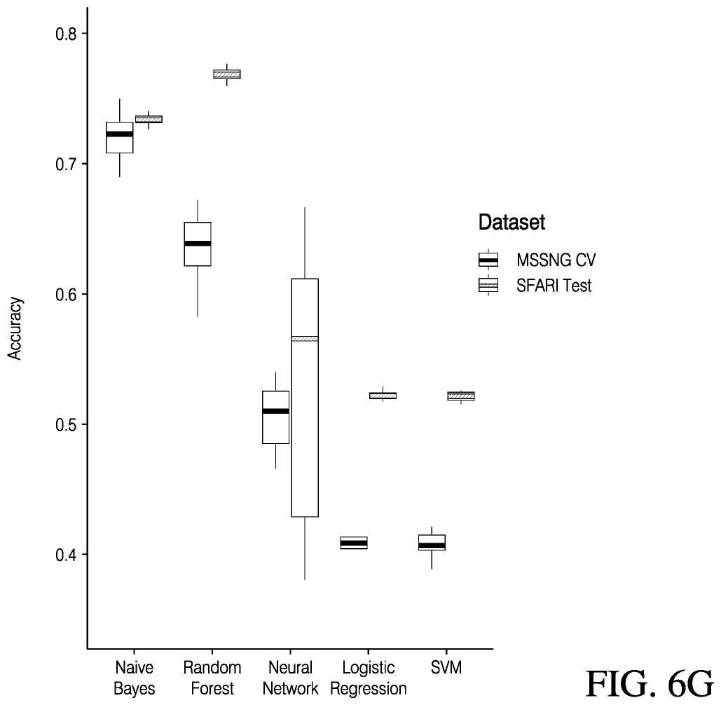

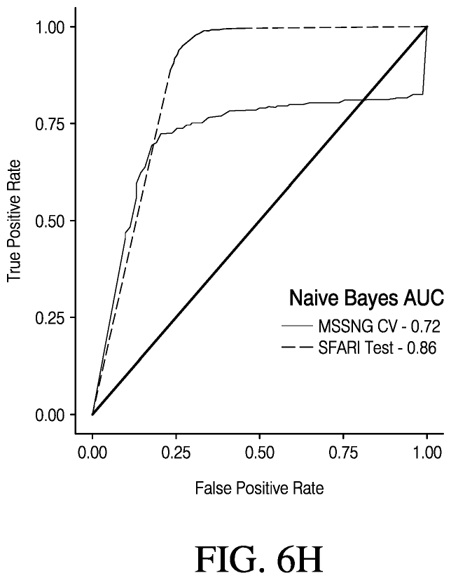

FIG. 6A illustrates a vector pre-processing and training scheme 600, according to various embodiments. Any one of multiple machine learning models may be used to classify variant burden vectors (e.g., as in the rows of the variant burden matrix, see FIG. 4). In various embodiments, the vector dimensionality of original vector 610 is halved (e.g., to 15,338 genes) to reduced vector 620, as illustrated above, by using only the top half of the scores from the variant burden matrix. In some embodiments, using 10-fold cross-validation, iteratively, 90% of the vectors 630 are chosen for training model 640, and the remaining 10% are tested 650 to calculate performance measures.

Classifiers may be trained using, without limitation, any one or more of multiple models such as: logistic regression (LR), support vector machines (SVM), multilayer perceptron (neural network), Naive Bayes, and Random Forest. Other models may be used, according to accuracy and efficacy. The training of each of these models includes predicting the disease/control status of a sample given its variant burden vector. In various embodiments, the model includes a multilayer neural network, wherein each of the layers includes a node coupled through a non-linear relation with one or more nodes in adjacent layers, or in the same layer. The non-linear relation includes model coefficients adjusted according to a feedback iteration process, or training. The feedback for the model coefficients is positive when the model correctly predicts the sample status (e.g., healthy/disease), and negative when the prediction is wrong.

For each model, training may include cross-validation such as a k-fold cross-validation procedure or leave-p-out cross-validation. Cross-validation, which may also be called rotation estimation or out-of-sample testing, may include any of various similar model validation techniques for assessing how the results of a statistical analysis will generalize to an independent data set. In k-fold cross-validation, the original sample may be randomly partitioned into k equal sized subsamples. Of the k subsamples, a single subsample may be retained as the validation data for testing the model, and the remaining k−1 subsamples may be used as training data. The cross-validation process may then be repeated k times, with each of the k subsamples used exactly once as the validation data. The k results can then be averaged to produce a single estimation. For example, a 10-fold cross-validation procedure may include iteratively using 90% of the reduced vector data for training and 10% of the reduced vector data for testing, then rotating the segments of data used for training/testing. In other embodiments, leave-p-out cross-validation (LpO CV) may be applied by using a pre-selected number of observations (e.g., p observations) as the validation set and the remaining observations as the training set. This may be repeated on all ways to cut the original sample on a validation set of p observations and a training set.

Various embodiments may select a classification model based on the accuracy of the predicted results after a number of iterations of the feedback loop. In various embodiments, the accuracy is measured as a fraction of correct case-control (e.g., disease-healthy) predictions on the test data. In various embodiments, a classification model may be selected from receiver operating curves obtained with test data on the trained models. The receiver curve plots the number of ‘True’ positive assessments (e.g., disease is present, as predicted) vs. the number of ‘False’ positives (e.g., disease is predicted but not present) to assess the robustness of balancing sensitivity and specificity for each model. It may be desirable that the receiver curve be a step function climbing from 0 to 1 in the ordinates (True Positive) when the abscissa is zero (False Positive). Accordingly, in various embodiments, the classification model of choice may be the one that renders the more ideal receiver curve.

FIG. 6B illustrates average model accuracy across cross-validation folds in a set of genomics data from the MSSNG database. Five different classification models, logistic regression (LR), support vector machines (SVM), multilayer perceptron (neural network), Naive Bayes, and Random Forest, were trained, and each demonstrated high mean accurate across all cross-validation folds, ranging between 85% and 95%. Logistic regression, SVM, and the artificial neural network exceeded an average of 90% accuracy. Of these three, the SVM model had the least variance across folds (93±0.005%, mean±SD accuracy across folds).

FIG. 6C illustrates classifier sensitivity and specificity of five different classification models through a representative receiver operating curve. For the last fold, receiver operating curves were calculated to show the trade-off between model sensitivity and specificity. Area under the receiving operating characteristic curve (AUROC), a performance measure of binary classification, is also listed in the legend for each model. The classifier curves are demonstrated with the logistic regression, SM, and neural network with AUROC as 0.966, 0.964, and 0.964, respectively. The neural network and Naïve Bayes have AUROC as 0.894 and 0.887, respectively. The black straight curve represents a random classifier. All five classifiers were able to attain a high ASD detection rate, while controlling for misclassification of control vectors.

FIG. 6D illustrates classification performance of five classification models in a second whole genome sequence dataset of independent ASD genomics data from the SFARI Simons Simplex Collection (SSC) in terms of average model accuracy. The set of genomics data was used to validate the genome vectorization and classification methodology. The set of genomics data includes healthy sibling controls, which may increase the complexity of the learning problem and impact performance. High accuracy was obtained by random forest, SVM, and logistic regression.

FIG. 6E illustrates classification performance of five classification models in another set of independent ASD genomics data (SFARI SSC) in terms of classifier sensitivity and specificity through a representative receiver operating curve. The set of genomics data is the same as in FIG. 6D.

FIGS. 6F-6H illustrate classification of ASD vectors using both MSSNG vectors and SFARI vectors to minimize class bias. The primary dataset used in this study excluded variants found only in controls, thus facilitating classification performance. Models were retrained using datasets that included all filtered variants from both ASD cases and controls, thus mitigating class bias.

FIG. 6F illustrates classification preprocessing vectors using both MSSNG vectors and SFARI vectors. Variant burden vectors were transformed using batch correction and principal component analysis (PCA) with inclusion of 99% of data variance. The MSSNG data was used for training classification models with 10-fold cross-validation, and the SFARI data was used exclusively for testing.

FIG. 6G illustrates average model accuracy of five classification models using both MSSNG vectors and SFARI vectors. Five different classification models were tested, but only the Naive Bayes model consistently performed well in both cross-validation and testing (CV: 72±1.8% and Test: 73±0.4%, mean±SD).

FIG. 6H illustrates the receiver operating characteristic curves of the Naive Bayes model using both MSSNG vectors and SFARI vectors. FIG. 6H shows balanced model sensitivity and specificity of the Naive Bayes model. The cross-validation and testing curve are specified in the legend, and the black curve represents a random classier. Area under the receiving operating characteristic curve (AUC), a performance measure of binary classification, is also listed in the legend for each model.

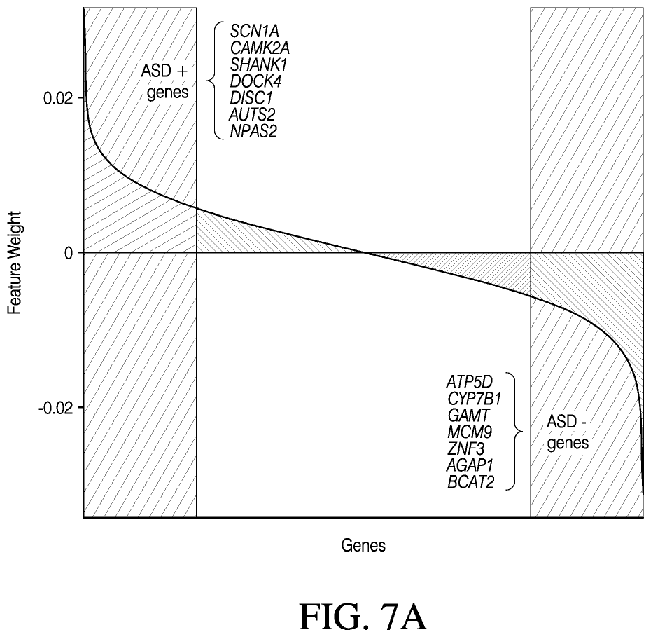

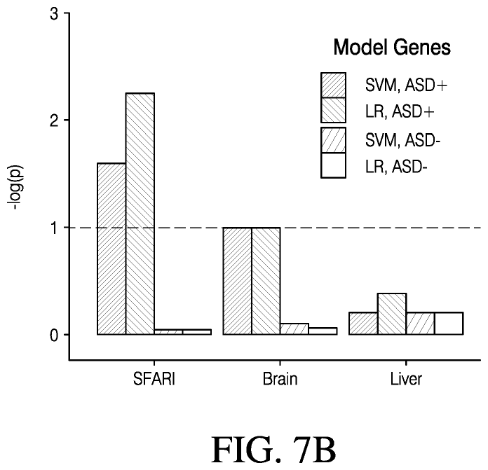

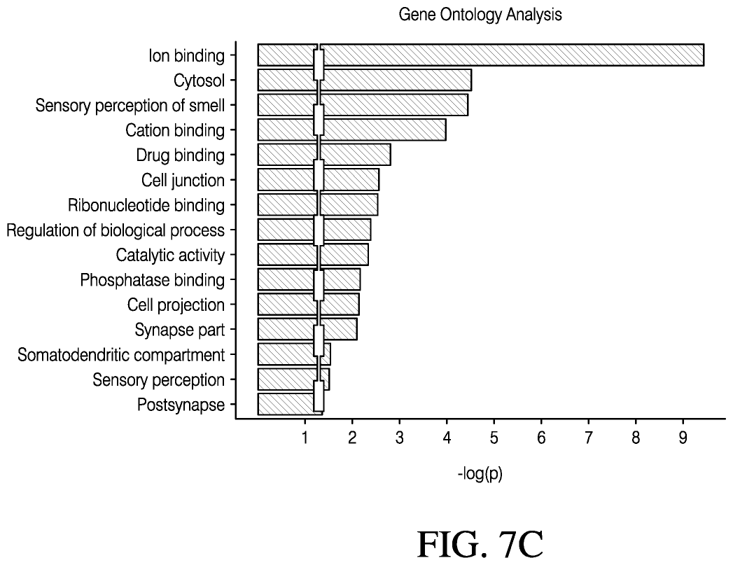

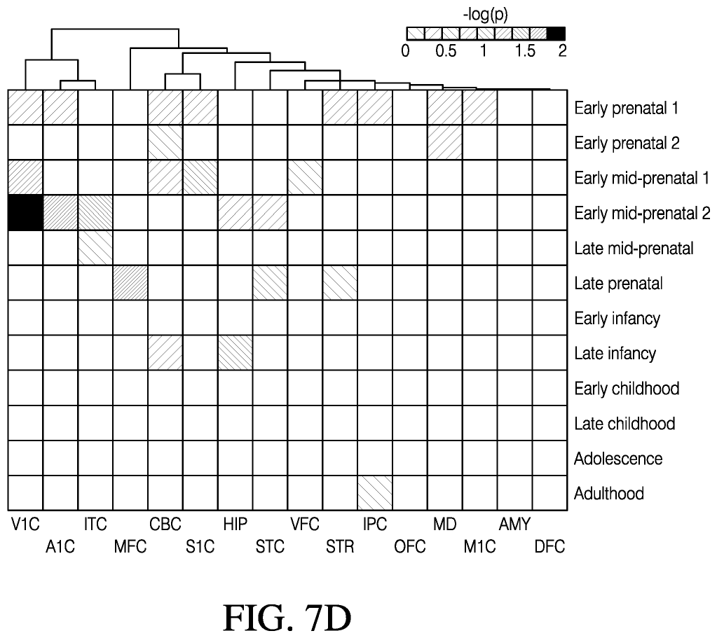

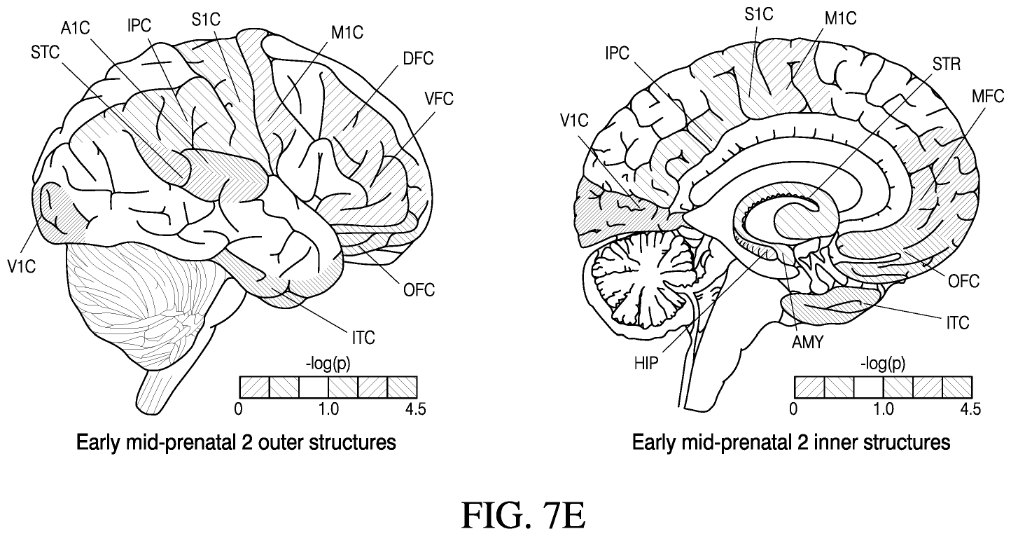

FIGS. 7A-E illustrate the extraction of salient genes for an exemplary hereditary disease (e.g., ASD), and the biological relevance of the extracted salient genes, according to various embodiments. A genome-wide ranking was assigned for each gene, based on the hyperplane weights learned by the model during training. The top and bottom quintile (SVM, ASD+ and SVM, ASD−) genes were chosen as representative gene lists for ASD relevant and ASD irrelevant genes, respectively. Both of these lists contained 3,067 genes (e.g., 15,338/5). In a similar manner, top and bottom quintile genes were selected from the logistic regression model (LR, ASD+ and LR, ASD). These lists were compared to existing sets of putative ASD genes (Princeton), evidence-based ASD genes (SFARI), and highly expressed brain genes using the binomial test for overrepresentation. Significance was set at p≤0.10, for each test, where p is the probability that the coincidence between the identified genes with the putative gene sets was purely random in a normal distribution (e.g., Fisher's exact test). In various embodiments, a set of highly expressed genes in the human liver may be included as a negative control. This may be the case in the understanding that genes expressed in the human liver may have little to no effect in ASD symptomatology or causality.

FIG. 7A illustrates a quintile plot of SVM hyperplane weights. The top and bottom quintile classifier genes, ASD+ and ASD−, respectively, are selected according to the variant scoring (see FIG. 4 for example). The ASD+ list includes genes deemed to be important for ASD classification, and the ASD− list are attributed to the control class. For example, the presence of the ASD+ gene in a patient's genome may enhance the likelihood that the person suffers from (or will suffer at some point) the hereditary disease. On the other hand, the presence of the ASD− genes in a patient's genome may increase the likelihood that the patient does not suffer from the hereditary disease. Both lists contain 3,067 genes (=1/5 of initial 30,729/2−top performers, see FIG. 6 for example). The quintile plot shows the genome-wide rankings and some representative genes from each list (SVM, ASD+ and SVM, ASD−) for the SVM model. A similar procedure may be performed for the logistic regression (LR) model to create LR, ASD+ and LR, ASD− lists of selected genes.

FIG. 7B illustrates a bar chart of ASD+ classifier genes enriched for ASD and brain related gene sets, according to different putative databases. More specifically, the ASD+ and ASD− lists were tested for overlap with a set of genome-wide putative ASD genes (Princeton), experimentally validated ASD genes (SFARI), brain expressed genes, and liver expressed genes. Enrichment was calculated using a binomial test (e.g., Fisher's exact test) with a p-value cutoff of p=0.10 (−log(p)=1). Two sets of ASD+ lists were enriched in the Princeton, SFARI, and brain expressed genes, namely those corresponding to the SVM model and to the LR model, respectively. The figure demonstrates that, according to various embodiments, the ASD− genes were not enriched in any of these sets, as expected. Also, according to various embodiments, neither ASD+, nor ASD−, lists were enriched for liver genes, as expected.

FIG. 7C illustrates a gene ontology analysis suggesting plausible pathway involvement, according to various embodiments. To obtain the bar graph, SVM, ASD+ genes were tested for significant overlap with biological pathways, molecular functions, and cellular components using a false discovery rate corrected Fisher's exact test. Significance was determined by a p-value ≤0.05. A portion of relevant results are shown in the plot, including ion binding, synaptic, and sensory perception terms. The SVM, ASD+ genes were further studied with the Panther Database online tool to identify biological processes, molecular functions, and cellular components involved with the selected list. Fisher's test with false discovery rate correction was used to identify significantly enriched modules.

The results in FIGS. 7D-E are determined using a permutation testing technique to estimate spatiotemporal enrichment of the ASD+ sets. In various embodiments, gene rankings derived from the classification model (e.g., SVM) can be set to an exponential scale, as follows: the topmost gene is assigned a value of 1, and the bottommost gene is assigned a value close to 0. The difference in the average rank for the jth region-stage's gene list and a random gene list is calculated (d_{obs}). The region-stage gene list and random gene list can be shuffled, for example, 100,000 times, and the average difference between the two lists can be calculated to build the distribution of possible dₚₑᵣₘ values. Finally, the p-value of the d_{obs} can be calculated using the z-score derived from the dₚₑᵣₘ distribution. P-values can be adjusted for false discovery rate control, and significance is assigned to adjusted p-values ≤0.10.

Spatiotemporal enrichment of the SVM, ASD+ genes can be assessed using, for example, gene expression data from the BrainSpan Atlas of the Developing Human Brain. Normalized gene transcript counts were acquired for brain samples that varied across multiple time points and neuroanatomical regions. Twelve developmental stages, ranging from early prenatal to adulthood were included. Regionally, sixteen discrete brain structures were included, namely: primary visual cortex (V1C), primary auditory cortex (A1C), inferior temporal cortex (ITC), medial frontal cortex (MFC), cerebellar cortex (CBC), primary somatosensory cortex (S1C), hippocampus (HIP), superior temporal cortex (STC), ventral frontal cortex (VFC), striatum (STR), inferior parietal cortex (IPC), olfactory cortex (OFC), mediodorsal nucleus of thalamus (MD), primary motor cortex (M1C), amygdala (AMY), and dorsal frontal cortex (DFC).

In various embodiments, representative gene sets were chosen for each region-stage pair by calculating the modified z-score of a given gene in the distribution of counts for all region-stage pairs. The modified z-score is calculated using the median and median absolute deviation (MAD) in lieu of the average and standard deviation, because the median provides a better measure of centrality for the counts, which may not be normally distributed. The formula for the modified z-score for the ith gene and jth region-stage pair is given here:

$${z}_{i , j} = \frac{0 \text{.645} \cdot ( {count}_{i , j} - m e d i a {n}_{i} )}{M A {D}_{i}}$$

For the jth region-stage, genes for which z_{i,j}≥2 may be selected as representative genes, according to various embodiments.

FIG. 7D illustrates ASD+ classifier genes that are enriched for early midfetal cortical regions during development, according to various embodiments. Using gene expression data from the BrainSpan Atlas of the Developing Human Brain, the SVM, ASD+ signature was localized early mid-prenatal development (13-18 post-conceptional weeks—pcw−). During this developmental stage, cortical regions were found to be enriched for the selected ASD+ signature, specifically, the V1C, A1C, ITC, and S1C. In this heat map, the inverse log of the adjusted p-values are shown, after correction for false discovery rate. The grayed-out cells in the heat map correspond to brain structures absent in the early fetal brain.

FIG. 7E illustrates neuroanatomical visualization of putative ASD brain regions. To give a regional demonstration of the ASD+ signature, in situ, the raw permutation test p-values were plotted for the developmental stage with the most significance, early mid-prenatal 2 (16-18 pcw). Diffuse cortical involvement is apparent. However, interior structures, such as HIP and AMY are also enriched for the ASD+ genes.

### Diagnosis of Hereditary Diseases or Traits

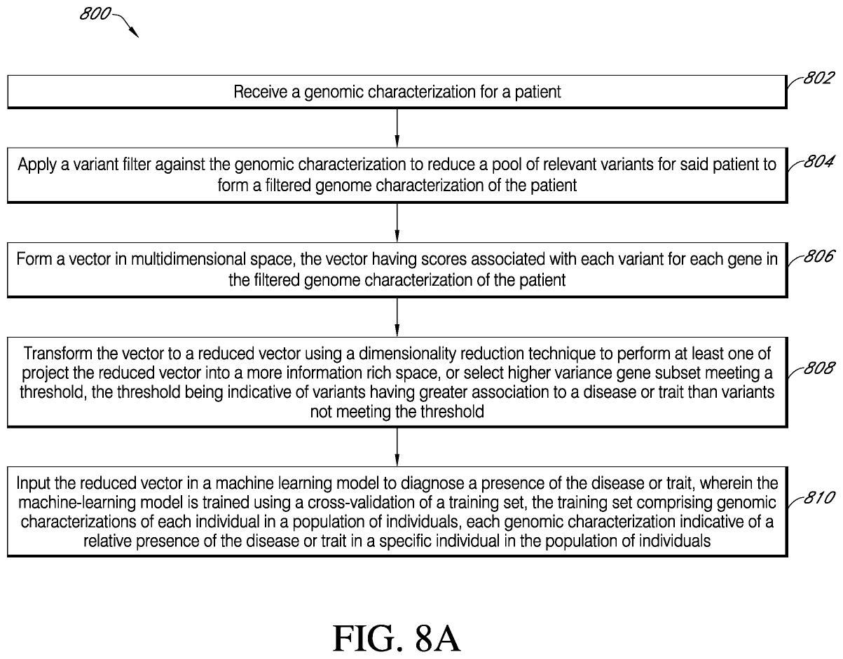

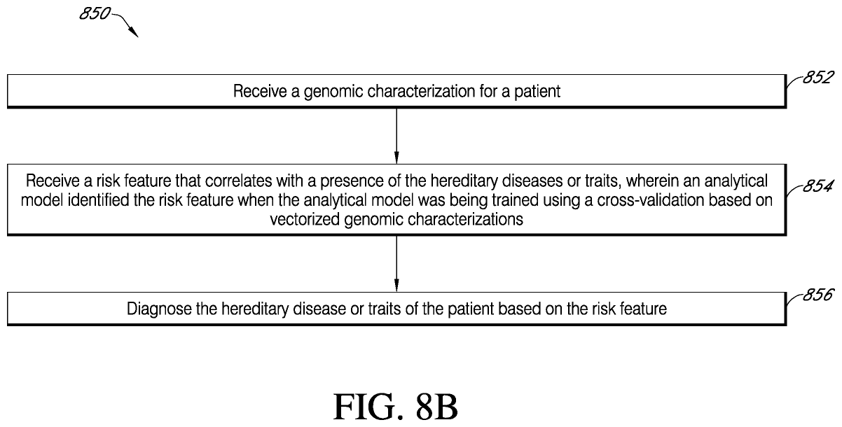

FIG. 8A is a flow chart illustrating steps in a method 800 for hereditary disease risk or trait assessment from a genetic characterization of an individual, according to various embodiments. Method 800 may be performed by one or more computers. In various embodiments, method 800 may be performed at least partially by any one of a plurality of servers in a network. For example, at least some of the steps in method 800 may be performed by one component in a mobile device running code for an application to access a remote server, or a component in the remote server. Accordingly, at least some of the steps in method 800 may be performed by a processor executing commands stored in a memory of one or more servers or the mobile device, or accessible by the server or the mobile device. Further, in various embodiments, at least some of the steps in method 800 may be performed overlapping in time, almost simultaneously, or in a different order from the order illustrated in method 800. Moreover, a method consistent with various embodiments disclosed herein may include at least one, but not all, of the steps in method 800.

Step 802 includes receiving a genomic characterization for a patient.

Step 804 includes applying a variant filter against the genomic characterization to reduce a pool of relevant variants for the patient to form a filtered genome characterization of the patient. In various embodiments, step 804 includes applying a raw filter based on a frequency of a variant being lower than a pre-selected value, a predicted damage of the variant, a documented association of the variant with clinical relevance, or on a salient annotation regarding the variant. In various embodiments, step 804 includes scoring a variant: a modifier, a low, a moderate, or a high consequence variant relative to the disease or trait based on an ensemble variant effect predictor algorithm.

Step 806 includes forming a vector in multidimensional space, the vector having scores associated with each variant for each gene in the filtered genome characterization of the patient.

Step 808 includes transforming the vector to a reduced vector using a dimensionality reduction technique to perform at least one of: project the reduced vector into a more information rich space, or select higher variance gene subset meeting a threshold. The threshold is indicative of variants having greater association to a disease or trait than variants not meeting the threshold. In various embodiments, step 808 includes using one of a principal component analysis technique or a t-distributed, stochastic neighbor embedded technique.

Step 810 includes inputting the reduced vector in a machine-learning model to diagnose a presence of the disease or trait, wherein the machine-learning model is trained using a cross-validation of a training set, the training set comprising genomic characterizations indicative of a relative presence of the disease or trait in a specific individual in the population of individuals. In various embodiments, step 810 includes identifying a risk feature in the reduced vector, the risk feature comprising one or more genes, variants, or transformed features indicative of a phenotypical manifestation of the disease or trait in the patient. In various embodiments, step 810 includes determining a presence of a disease in the patient, and determining a confidence level for the presence of the disease in the patient. In various embodiments, step 810 includes determining a discrete value such as disease presence or a continuous value indicative of a likelihood of the disease or a magnitude of the trait, further comprising identifying a range of the continuous value indicative of a confidence level for the continuous value. In various embodiments, the disease or trait includes one of autism, a neuropsychiatric disorder, or a neurotypical control, and step 810 includes quantifying a genetic risk of one of autism, a neuropsychiatric disorder, or a lack thereof. In various embodiments, step 810 includes identifying driver factors in the disease or trait based on a molecular correspondence with at least one component of the reduced vector. In various embodiments, step 810 includes identifying a subtype of the disease or trait by inputting the reduced vector in a clustering algorithm. In various embodiments, step 810 includes identifying an organ in the patient associated with the disease or trait based on gene expression of the gene associated with a component of the reduced vector. In various embodiments, step 810 includes identifying a treatment for the disease in the patient in correspondence with at least one component of the reduced vector and based on the presence of the disease or trait. In various embodiments, step 810 includes identifying at least one neuroanatomical region associated with the disease or trait based on a gene expression of the genes associated with the reduced vector.

FIG. 8B is a flow chart illustrating steps in a method 850 for hereditary disease risk or trait assessment from a genetic characterization of an individual, according to various embodiments. Method 850 may be performed by one or more computers. In various embodiments, method 850 may be performed at least partially by any one of a plurality of servers in a network. For example, at least some of the steps in method 850 may be performed by one component in a mobile device running code for an application to access a remote server, or a component in the remote server. Accordingly, at least some of the steps in method 850 may be performed by a processor executing commands stored in a memory of one or more servers or the mobile device, or accessible by the server or the mobile device. Further, in various embodiments, at least some of the steps in method 850 may be performed overlapping in time, almost simultaneously, or in a different order from the order illustrated in method 850. Moreover, a method consistent with various embodiments disclosed herein may include at least one, but not all, of the steps in method 850.

Step 852 includes receiving a genomic characterization for a patient.

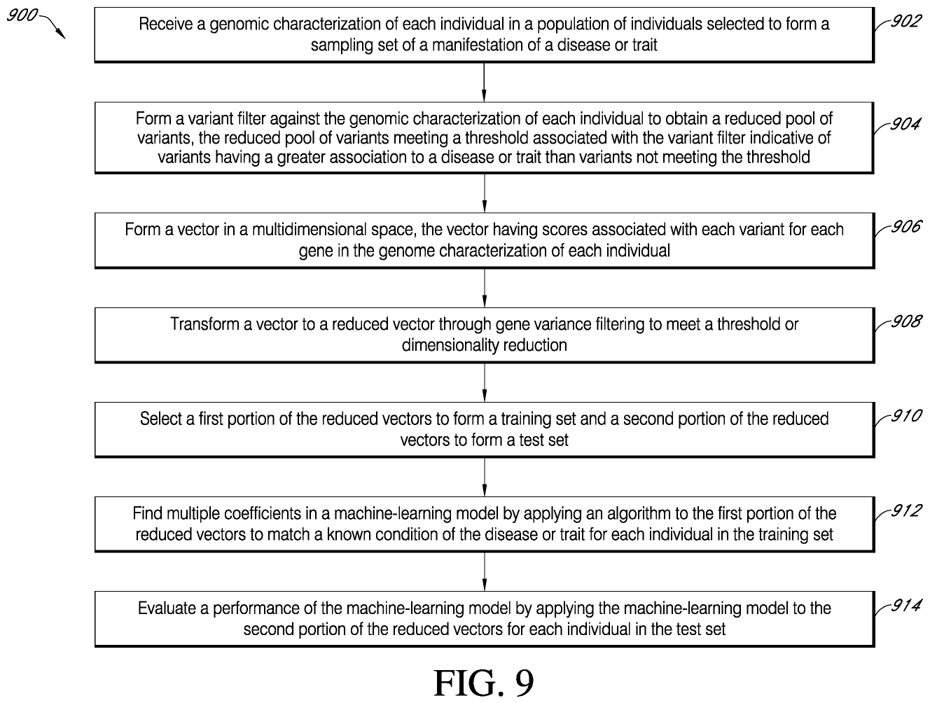

Step 854 includes receiving a risk feature that correlates with a presence of the hereditary diseases or traits, wherein an analytical model identified the risk feature when the analytical model was being trained using a cross-validation based on vectorized genomic characterizations. The training set and the validation set are portions of vectorized genomic characterizations of each individual in a population of individuals with a known presence or absence of the hereditary disease or traits. Step 854 can implement one or more steps in FIG. 9 to train one or more analytical models to obtain risk features associated with the hereditary diseases or traits to be diagnosed.

Step 856 includes diagnosing the hereditary disease or traits of the patient based on the risk feature.

Method 850 may further comprise receiving a plurality of genomic characterizations of each individual in the population of individuals; applying a variant filter against the genomic characterizations to reduce a pool of relevant variants to form a filtered genomic characterization; forming a vector in a multidimensional space, the vector including a score associated with each variant for each gene in the filtered genomic characterization for each individual in the population of individuals; transforming the vector to a reduced vector using a dimensionality reduction technique, the dimensionality reduction technique comprising one of a visualization tool for differentiating a vector projection in a reduced dimensional space according to a pre-selected boundary, or a selection of a higher variance gene subset meeting a pre-selected threshold; and inputting the reduced vector as the vectorized genomic characterizations in an analytical model to train the analytical model and to identify the risk feature.

In various embodiments, transforming the vector to a reduced vector comprises using one of a principal component analysis technique or a t-distributed, stochastic neighbor embedded technique.

In various embodiments, applying a variant filter against the genomic characterization to obtain a reduced pool of variants comprises applying a raw filter based on a frequency of a variant being lower than a pre-selected value, a predicted damage of the variant, a documented association of the variant with clinical relevance, or on a salient annotation regarding the variant or scoring a variant as one of: a modifier, a low, a moderate, or a high consequence variant, relative to the hereditary diseases or traits for each gene and each individual in the population of individuals.

In various embodiments, inputting the reduced vector in an analytical model comprises identifying the risk feature in the reduced vector, the risk feature comprising one or more genes, variants, or transformed features indicative of a phenotypical manifestation of the hereditary diseases or traits in the patient.

In various embodiments, inputting the reduced vector in an analytical model comprises applying one of a clustering model or a

In various embodiments, inputting the reduced vector in an analytical model comprises inputting the reduced vector in a machine learning model.

### Training of Analytical Models

FIG. 9 is a flow chart illustrating steps in a method for training an analytical model for risk assessment of hereditary diseases or traits, according to various embodiments. Method 900 may be performed by one or more computers. In various embodiments, method 900 may be performed at least partially by any one of a plurality of servers in a network. For example, at least some of the steps in method 900 may be performed by one component in a mobile device running code for an application to access a remote server, or a component in the remote server. Accordingly, at least some of the steps in method 900 may be performed by a processor executing commands stored in a memory of one or more servers or the mobile device, or accessible by the server or the mobile device. Further, in various embodiments, at least some of the steps in method 900 may be performed overlapping in time, almost simultaneously, or in a different order from the order illustrated in method 900. Moreover, a method consistent with various embodiments disclosed herein may include at least one, but not all, of the steps in method 900.

Step 902 includes receiving a genomic characterization of each individual in a population of individuals selected to form a sampling set of a manifestation of a disease or trait.

Step 904 includes forming a variant filter against the genomic characterization of each individual to obtain a reduced pool of variants, the reduced pool of variants meeting a threshold associated with the variant filter indicative of variants having a greater association to a disease or trait than variants not meeting the threshold. In various embodiments, step 904 includes applying a raw filter based on a frequency of a variant being lower than a pre-selected value, a predicted damage of the variant, a documented association of the variant with clinical relevance, or other salient annotations regarding the variant. In various embodiments, step 904 includes selecting a variant that may have an association with the disease or trait in the population of individuals. In various embodiments, step 904 includes applying a variant effect predictor algorithm to the filtered variants.

Step 906 includes forming a vector in a multidimensional space, the vector having scores associated with each variant for each gene in the genome characterization of each individual.

Step 908 includes transforming a vector to a reduced vector through gene variance filtering to meet a threshold or dimensionality reduction.

Step 910 includes selecting a first portion of the reduced vectors to form a training set and a second portion of the reduced vectors to form a validation set.

Step 912 includes finding multiple coefficients in a machine-learning model by applying an analytical model to the first portion of the reduced vectors to match a known condition of the disease or trait for each individual in the training set.

Step 914 includes evaluating a performance of the machine-learning model by applying the machine-learning model to the second portion of the reduced vectors for each individual in the validation set. In various embodiments, the population of individuals is selected according to multiple degrees of a phenotype for a disease or trait, and step 914 includes determining an algorithm for clustering the reduced vectors, according to a subtype of the disease or trait. In various embodiments, the known condition of the disease or trait includes, for a first individual, a heritable neuropsychiatric condition or trait, and step 914 includes selecting, in a genomic characterization of the first individual, a genomic sequence associated with multiple neuroanatomical regions. In various embodiments, the known condition of the disease or trait includes, for a first individual, a neuropsychiatric condition, and step 914 includes selecting, in a genomic characterization of the first individual, a genomic sequence associated with multiple developmental stages. In various embodiments, step 914 includes applying a spatiotemporal enrichment analysis to asses a development stage and a neuroanatomical region associated with the disease or trait. In various embodiments, step 914 includes scoring a variant as one of a modifier, a low, a moderate, or a high consequence variant relative to the disease or trait based on a variant effect predictor algorithm. In various embodiments, step 914 includes training a model with reduced vectors to select a risk feature from multiple components in the reduced vectors, the risk feature indicative of a phenotypical manifestation of the disease or trait for each individual in the sampling set of a relative manifestation of a disease or set.

### Computer System

In various embodiments, the methods for diagnosing hereditary diseases or traits or training an analytical model can be implemented via various systems such as computer software or hardware or a combination thereof.

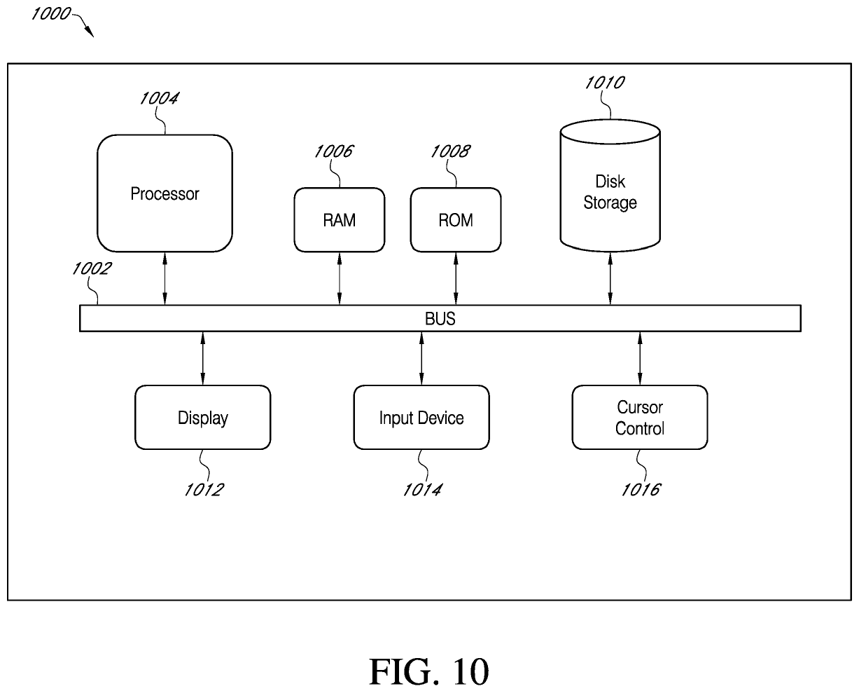

FIG. 10 is a block diagram that illustrates a computer system 1000, upon which embodiments, or portions of the embodiments, of the present teachings may be implemented. In various embodiments of the present teachings, computer system 1000 can include a bus 1002 or other communication mechanism for communicating information, and a processor 1004 coupled with bus 1002 for processing information. In various embodiments, computer system 1000 can also include a memory 1006, which can be a random access memory (RAM) or other dynamic storage device, coupled to bus 1002 for determining instructions to be executed by processor 1004. Memory 1006 also can be used for storing temporary variables or other intermediate information during execution of instructions to be executed by processor 1004. In various embodiments, computer system 1000 can further include a read-only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a trackball or cursor direction keys for communicating direction information and command selections to processor 1004 and for controlling cursor movement on display 1012. This input device 1014 typically has two degrees of freedom in two axes, a first axis (e.g., x) and a second axis (e.g., y), that allows the device to specify positions in a plane. However, it should be understood that input devices 1014 allowing for 3-dimensional (x, y, and z) cursor movement are also contemplated herein.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1010. Execution of the sequences of instructions contained in memory 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, and magnetic disks, such as storage device 1010. Examples of volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein including flow charts, diagrams, and accompanying disclosure can be implemented using computer system 1000 as a standalone device or on a distributed network of shared computer processing resources such as a cloud computing network.

In accordance with various embodiments, the systems and methods described herein can be implemented using computer system 1000 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Although specific embodiments and applications of the disclosure have been described in this specification (including the associated Appendix), these embodiments and applications are exemplary only, and many variations are possible.

### RECITATION OF EMBODIMENTS

1. A computer-implemented method to diagnose hereditary diseases or traits, comprising: receiving a genomic characterization for a patient; receiving a risk feature that correlates with a presence of the hereditary diseases or traits, wherein an analytical model identified the risk feature when the analytical model was being trained using a cross-validation of a training set and a validation set, the training set and the validation set are portions of vectorized genomic characterizations of each individual in a population of individuals with a known presence or absence of the hereditary disease or traits, and diagnosing the hereditary disease or traits of the patient based on the risk feature, wherein the presence of the hereditary diseases or traits of the patient is diagnosed when the genomic characterization for the patient indicates a presence of the risk feature.

2. The computer-implemented method of claim 1, further comprising receiving a plurality of genomic characterizations of each individual in the population of individuals; applying a variant filter against the genomic characterizations to reduce a pool of relevant variants to form a filtered genomic characterization; forming a vector in a multidimensional space, the vector including a score associated with each variant for each gene in the filtered genomic characterization for each individual in the population of individuals; transforming the vector to a reduced vector using a dimensionality reduction technique, the dimensionality reduction technique comprising one of a visualization tool for differentiating a vector projection in a reduced dimensional space according to a pre-selected boundary, or a selection of a higher variance gene subset meeting a pre-selected threshold; and inputting the reduced vector as the vectorized genomic characterizations in an analytical model to train the analytical model and to identify the risk feature.

3. The computer-implemented method of claim 2, wherein transforming the vector to a reduced vector comprises using one of a principal component analysis technique or a t-distributed, stochastic neighbor embedded technique.

4. The computer-implemented method of any of claims 2-3, wherein applying a variant filter against the genomic characterization to obtain a reduced pool of variants comprises applying a raw filter based on a frequency of a variant being lower than a pre-selected value, a predicted damage of the variant, a documented association of the variant with clinical relevance, or on a salient annotation regarding the variant, or scoring a variant as one of: a modifier, a low, a moderate, or a high consequence variant, relative to the hereditary diseases or traits for each gene and each individual in the population of individuals.

5. The computer-implemented method of any of claims 2-4, wherein inputting the reduced vector in an analytical model comprises identifying the risk feature in the reduced vector, the risk feature comprising one or more genes, variants, or transformed features indicative of a phenotypical manifestation of the hereditary diseases or traits in the patient.

6. The computer-implemented method of any of claims 2-5, wherein inputting the reduced vector in an analytical model comprises applying one of a clustering model or a regression model to the reduced vector.

7. The computer-implemented method of any of claims 2-6, wherein inputting the reduced vector in an analytical model comprises inputting the reduced vector in a machine learning model.

8. The computer-implemented method of any of claims 1-7, further comprising determining a presence of a disease in the patient, and determining a confidence level for the presence of the disease in the patient.

9. The computer-implemented method of any of claims 1-8, further comprising determining a discrete value such as disease presence or a continuous value indicative of a stage of the hereditary diseases or a magnitude of the hereditary diseases or traits, or further comprising identifying a range of the continuous value indicative of a confidence level for the continuous value.

10. The computer-implemented method of any of claims 2-9, further comprising identifying driver factors in the hereditary diseases or traits based on a molecular correspondence with at least one component of the reduced vector.

11. The computer-implemented method of any of claims 2-10, further comprising identifying a subtype of hereditary diseases or traits by inputting the reduced vector in a clustering algorithm.

12. The computer-implemented method of any of claims 2-11, further comprising identifying an organ in the patient associated with hereditary diseases or traits based on gene expression of the gene associated with a component of the reduced vector.

13. The computer-implemented method of any of claims 2-12, further comprising identifying a treatment for the hereditary diseases in the patient in correspondence with at least one component of the reduced vector and based on the presence of the hereditary diseases or traits.

14. The computer-implemented method of any of claims 2-13, further comprising identifying at least one neuroanatomical region associated with the hereditary diseases or traits based on a gene expression of the risk feature associated with the reduced vector.

15. The computer-implemented method of any of claims 1-14, wherein the hereditary diseases or traits comprises one of autism, a neuropsychiatric disorder, or a neurotypical control, and diagnosing the hereditary diseases or traits comprises diagnosing one of autism, a neuropsychiatric disorder, or a lack thereof.

16. A system for a diagnostic of hereditary diseases or traits, comprising: a memory storing instructions; and one or more processors configured to execute the instructions to cause the system to: receive a genomic characterization for a patient; apply a variant filter against the genomic characterization to obtain a reduced pool of variants, the reduced pool of variants comprising a higher subset of rare, damaging, or otherwise relevant variants indicative of variants having greater association to a disease or trait than variants not meeting a threshold; form a vector in a multidimensional space, the vector having scores associated with each variant for each gene in the genome characterization of the patient; transform the vector to a reduced vector based on a visualization tool for differentiating a vector projection in a reduced dimensional space according to a pre-selected boundary, or on a higher variance gene subset meeting a threshold; and input the reduced vector in an analytical model for the diagnostic of hereditary diseases or traits, wherein the analytical model is trained using a cross-validation of a training set, the training set comprising genomic characterizations of each individual in a population of individuals, each genomic characterization indicative of a relative presence of hereditary diseases or traits in a specific individual in the population of individuals.

17. The system of claim 16, wherein to apply a variant filter against the genomic characterization to reduce a pool of relevant variants the one or more processors execute instructions to score a variant as one of: a modifier, a low, a moderate, or a high consequence variant, relative to the disease or trait.

18. The system of embodiment 16, wherein to input the reduced vector in an analytical model for the diagnostic of hereditary diseases or traits, the one or more processors execute instructions to determine a presence of a disease in the patient, and to determine a confidence level for the presence of the disease in the patient.

19. The system of embodiment 16, wherein for the diagnostic of hereditary diseases or traits, the one or more processors execute instructions to determine a continuous value, the continuous value being indicative of hereditary diseases or a magnitude of the traits, and the one or more processors execute instructions to identify a range of the continuous value indicative of a confidence level for the continuous value.

20. A computer-implemented method to train an analytical model for diagnosis of hereditary diseases or traits, comprising: receiving a genomic characterization of each individual in a population of individuals, the population of individuals selected to form a sampling set of a relative manifestation of a disease or trait; forming a variant filter against the genomic characterization of each individual to obtain a reduced pool of variants, the reduced pool of variants meeting a threshold associated with the variant filter, indicative of variants having a greater association to a disease or trait than variants not meeting the threshold; forming a vector in a multidimensional space, the vector having scores associated with each variant for each gene in the genome characterization of each individual; transforming a vector to a reduced vector through gene variance filtering to meet a threshold or dimensionality reduction; selecting a first portion of the reduced vectors, to form a training set and a second portion of the reduced vectors, to form a validation set; finding multiple coefficients in an analytical model by applying the analytical model to the first portion of the reduced vectors to match a known condition of the disease or trait for each individual in the training set; and evaluating a performance of the analytical model by applying the analytical model to the second portion of the reduced vectors for each individual in the validation set.

21. The computer-implemented method of embodiment 20, wherein forming a variant filter against the genomic characterization of each individual to obtain a reduced set of variants comprises applying a raw filter based on a frequency of a variant being lower than a pre-selected value, a predicted damage of the variant, a documented association of the variant with clinical relevance, or other salient annotations regarding the variant.

22. The computer-implemented method of embodiment 20 or 21, wherein scoring filtered variants to obtain a vector comprises scoring a variant as one of: a modifier, a low, a moderate, or a high consequence variant relative to the disease or trait based on a variant effect predictor algorithm.

23. The computer-implemented method of any one of embodiments 20 to 22, wherein forming a variant filter comprises selecting a variant that may have an association with the disease or trait in the population of individuals.

24. The computer-implemented method of any one of embodiments 20 to 23, wherein training a model with reduced vectors enables selecting a risk feature from multiple components in the reduced vectors, the risk feature indicative of a phenotypical manifestation of the disease or trait for each individual in the sampling set of a relative manifestation of a disease or set.

25. The computer-implemented method of any one of embodiments 20 to 24, wherein the population of individuals is selected according to multiple degrees of a phenotype for a disease or trait, the method further comprising determining an algorithm for clustering the reduced vectors, according to a subtype of the disease or trait.

26. The computer-implemented method of any one of embodiments 20 to 25, wherein forming a variant scorer comprises applying a variant effect predictor algorithm to the filtered variants.

27. The computer-implemented method of any one of embodiments 20 to 26, wherein the known condition of the disease or trait includes, for a first individual, a neuropsychiatric condition, further comprising selecting, in a genomic characterization of the first individual, a genomic sequence associated with multiple developmental stages.

28. The computer-implemented method of any one of embodiments 20 to 26, wherein the known condition of the disease or trait includes, for a first individual, a heritable neuropsychiatric condition or trait, further comprising selecting, in a genomic characterization of the first individual, a genomic sequence associated with multiple neuroanatomical regions.

29. The computer-implemented method of any one of embodiments 20 to 28, further comprising applying a spatiotemporal enrichment analysis to asses a development stage and a neuroanatomical region associated with the disease or trait.

30. The computer-implemented method of claim 20, wherein the analytical model is logistic regression, support vector machine, multilayer perceptron, Naïve Bayes, random forest, or a combination thereof.

What is claimed is:

1. A computer-implemented method to diagnose hereditary diseases or traits, comprising:
receiving a genomic characterization for a patient;
receiving a risk feature that correlates with a presence of the hereditary diseases or traits, wherein an analytical model identified the risk feature when the analytical model was being trained using a cross-validation of a training set and a validation set, the training set and the validation set are portions of vectorized genomic characterizations of each individual in a population of individuals with a known presence or absence of the hereditary disease or traits; and
diagnosing the hereditary disease or traits of the patient based on the risk feature, wherein the presence of the hereditary diseases or traits of the patient is diagnosed when the genomic characterization for the patient indicates a presence of the risk feature.

2. The computer-implemented method of claim 1, further comprising
receiving a plurality of genomic characterizations of each individual in the population of individuals;
applying a variant filter against the genomic characterizations to reduce a pool of relevant variants to form a filtered genomic characterization;
forming a vector in a multidimensional space, the vector including a score associated with each variant for each gene in the filtered genomic characterization for each individual in the population of individuals;
transforming the vector to a reduced vector using a dimensionality reduction technique, the dimensionality reduction technique comprising one of a visualization tool for differentiating a vector projection in a reduced dimensional space according to a pre-selected boundary, or a selection of a higher variance gene subset meeting a pre-selected threshold; and
inputting the reduced vector as the vectorized genomic characterizations in an analytical model to train the analytical model and to identify the risk feature.

3. The computer-implemented method of claim 2, wherein transforming the vector to a reduced vector comprises using one of a principal component analysis technique or a t-distributed, stochastic neighbor embedded technique.

4. The computer-implemented method of claim 2, wherein applying a variant filter against the genomic characterization to obtain a reduced pool of variants comprises applying a raw filter based on a frequency of a variant being lower than a pre-selected value, a predicted damage of the variant, a documented association of the variant with clinical relevance, or on a salient annotation regarding the variant or scoring a variant as one of: a modifier, a low, a moderate, or a high consequence variant, relative to the hereditary diseases or traits for each gene and each individual in the population of individuals.

5. The computer-implemented method of claim 2, wherein inputting the reduced vector in an analytical model comprises identifying the risk feature in the reduced vector, the risk feature comprising one or more genes, variants, or transformed features indicative of a phenotypical manifestation of the hereditary diseases or traits in the patient.

6. The computer-implemented method of claim 2, wherein inputting the reduced vector in an analytical model comprises applying one of a clustering model or a regression model to the reduced vector.

7. The computer-implemented method of claim 2, wherein inputting the reduced vector in an analytical model comprises inputting the reduced vector in a machine learning model.

8. The computer-implemented method of claim 1, further comprising determining a presence of a disease in the patient, and determining a confidence level for the presence of the disease in the patient.

9. The computer-implemented method of claim 1, further comprising determining a discrete value such as disease presence or a continuous value indicative of a stage of the hereditary diseases or a magnitude of the hereditary diseases or traits, or further comprising identifying a range of the continuous value indicative of a confidence level for the continuous value.

10. The computer-implemented method of claim 2, further comprising identifying driver factors in the hereditary diseases or traits based on a molecular correspondence with at least one component of the reduced vector.

11. The computer-implemented method of claim 2, further comprising identifying a subtype of hereditary diseases or traits by inputting the reduced vector in a clustering algorithm.

12. The computer-implemented method of claim 2, further comprising identifying an organ in the patient associated with hereditary diseases or traits based on gene expression of the gene associated with a component of the reduced vector.

13. The computer-implemented method of claim 2, further comprising identifying a treatment for the hereditary diseases in the patient in correspondence with at least one component of the reduced vector and based on the presence of the hereditary diseases or traits.

14. The computer-implemented method of claim 2, further comprising identifying at least one neuroanatomical region associated with the hereditary diseases or traits based on a gene expression of the risk feature associated with the reduced vector.

15. The computer-implemented method of claim 1, wherein the hereditary diseases or traits comprises one of autism, a neuropsychiatric disorder, or a neurotypical control, and diagnosing the hereditary diseases or traits comprises diagnosing one of autism, a neuropsychiatric disorder, or a lack thereof.

16. A system for a diagnosis of hereditary diseases or traits, comprising:
a memory storing instructions; and
one or more processors configured to execute the instructions to cause the system to:
receive a genomic characterization for a patient;
apply a variant filter against the genomic characterization to obtain a reduced pool of variants, the reduced pool of variants comprising a higher subset of rare, damaging, or otherwise relevant variants indicative of variants having greater association to a disease or trait than variants not meeting a threshold;
form a vector in a multidimensional space, the vector having scores associated with each variant for each gene in the genome characterization of the patient;
transform the vector to a reduced vector based on a visualization tool for differentiating a vector projection in a reduced dimensional space according to a pre-selected boundary, or on a higher variance gene subset meeting a threshold; and
input the reduced vector in an analytical model for identifying one or risk features related to the diagnosis of hereditary diseases or traits, wherein the analytical model is trained using a cross-validation of a training set, the training set comprising genomic characterizations of each individual in a population of individuals, each genomic characterization indicative of a relative presence of hereditary diseases or traits in a specific individual in the population of individuals, and
diagnose the patient based on a presence or an absence of the risk feature, wherein the genomic characterization for the patient having the risk feature indicates that the patient has the hereditary diseases or traits.

17. The system of claim 16, wherein to apply a variant filter against the genomic characterization to reduce a pool of relevant variants the one or more processors execute instructions to score a variant as one of: a modifier, a low, a moderate, or a high consequence variant, relative to the disease or trait.

18. The system of claim 16, wherein to diagnose the patient based on a presence or an absence of the risk feature, the one or more processors execute instructions to determine a confidence level for the presence of the hereditary diseases or traits in the patient.

19. The system of claim 16, wherein diagnose the patient based on a presence or an absence of the risk feature, the one or more processors execute instructions to determine a continuous value, the continuous value being indicative of hereditary diseases or a magnitude of the traits, and the one or more processors execute instructions to identify a range of the continuous value indicative of a confidence level for the continuous value.

20. A computer-implemented method to train an analytical model for diagnosis of hereditary diseases or traits, comprising:
receiving a genomic characterization of each individual in a population of individuals, the genomic characterizations comprising a pool of variants, the population of individuals selected to form a sampling set of a relative manifestation of a disease or trait;
forming a variant filter against the genomic characterization of each individual to obtain a reduced pool of variants, the reduced pool of variants meeting a threshold associated with the variant filter;
forming a vector in a multidimensional space using the reduced pool of variants, the vector having scores associated with each variant in the reduced pool of variants for each gene in the genome characterization of each individual;
transforming a vector to a reduced vector through a dimensionality reduction technique to reduce dimensionality of the vector;
training an analytical model with the reduced vector, wherein training the analytical model comprises selecting a first portion of the reduced vector to form a training set and a second portion of the reduced vector to form a validation set;
finding multiple coefficients in the analytical model by applying the analytical model to the first portion of the reduced vector to match a known condition of the disease or trait for each individual in the training set; and
evaluating a performance of the analytical model by applying the analytical model to the second portion of the reduced vector for each individual in the validation set.

21. The computer-implemented method of claim 20, wherein forming a variant filter against the genomic characterization of each individual to obtain a reduced set of variants comprises applying a raw filter based on a frequency of a variant being lower than a pre-selected value, a predicted damage of the variant, a documented association of the variant with clinical relevance, or other salient annotations regarding the variant.

22. The computer-implemented method of claim 20, wherein scoring reduced pool of variants to obtain a vector comprises scoring a variant as one of: a modifier, a low, a moderate, or a high consequence variant relative to the disease or trait based on a variant effect predictor algorithm.

23. The computer-implemented method of claim 20, wherein forming a variant filter comprises selecting a variant that may have an association with the disease or trait in the population of individuals.

24. The computer-implemented method of claim 20, wherein training the analytical model with the reduced vector further comprises selecting a risk feature from multiple components in the reduced vector, the risk feature indicative of a phenotypical manifestation of the disease or trait for each individual in the sampling set of a relative manifestation of a disease or set.

25. The computer-implemented method of claim 20, wherein the population of individuals is selected according to multiple degrees of a phenotype for a disease or trait, the method further comprising determining an algorithm for clustering the reduced vector, according to a subtype of the disease or trait.

26. The computer-implemented method of claim 20, wherein forming a variant scorer comprises applying a variant effect predictor algorithm to the reduced pool of variants.

27. The computer-implemented method of claim 20, wherein the known condition of the disease or trait includes, for a first individual, a neuropsychiatric condition, further comprising selecting, in a genomic characterization of the first individual, a genomic sequence associated with multiple developmental stages.

28. The computer-implemented method of claim 20, wherein the known condition of the disease or trait includes, for a first individual, a heritable neuropsychiatric condition or trait, further comprising selecting, in a genomic characterization of the first individual, a genomic sequence associated with multiple neuroanatomical regions.

29. The computer-implemented method of claim 20, further comprising applying a spatiotemporal enrichment analysis to asses a development stage and a neuroanatomical region associated with the disease or trait.

30. The computer-implemented method of claim 20, wherein the analytical model is selected from the group consisting of logistic regression, support vector machine, multilayer perceptron, Naïve Bayes, random forest, and a combination thereof.
